(19) United States
(12) Patent Application Publication
(10) Pub. No.: US 2024/0180537 A1
(43) Pub. Date: Jun. 6, 2024

(54) HANDLES FOR MEDICAL DEVICES

(71) Applicant: Boston Scientific Scimed, Inc., Maple Grove, MN (US)

(72) Inventor: Austin Grant JOHNSON, Worcester, MA (US)

(73) Assignee: Boston Scientific Scimed, Inc., Maple Grove, MN (US)

(21) Appl. No.: 18/524,156

(22) Filed: Nov. 30, 2023

Related U.S. Application Data
(60) Provisional application No. 63/429,262, filed on Dec. 1, 2022.

(51) Int. Cl. A61B 17/00

(52) U.S. Cl. CPC A61B 17/00234; A61B 2017/00327; A61B 2017/0034

(57) ABSTRACT

A medical device handle may include an actuator; a first pulley; and a second pulley. The second pulley may be movable relative to the first pulley along an axis extending between a first axle of the first pulley and a second axle of the second pulley. A pull member may be wrapped around the first pulley and the second pulley, and coupled to the actuator and a control member may be coupled to the second pulley. Movement of the second pulley along the axis may be configured to move the control member in a direction parallel to or coaxial with the axis.

### CROSS-REFERENCE TO RELATED APPLICATIONS

This application claims the benefit of priority under 35 U.S.C. § 119 from U.S. Provisional Application No. 63/429,262, filed Dec. 1, 2022, which is incorporated by reference herein in its entirety.

### TECHNICAL FIELD

The disclosure relates generally to handles for medical devices. More specifically, aspects of the disclosure pertain to handles for medical device handles that include a control assembly for providing a mechanical advantage.

### BACKGROUND

During a medical procedure, an operator may utilize a medical device that includes a handle and a shaft extending distally therefrom. For example, the medical device may be an endoscopic medical device. The shaft of the medical device may be inserted into a working channel of an endoscope (or other scope), advanced through the working channel, and extended out of a distal opening of the working channel, at a distal tip of the endoscope. An operator may actuate the medical device using the handle of the medical device. For example, the operator may activate an actuator at the handle. In an example, the actuator may include moving a finger grip.

In some examples, activating the actuator of a medical device may require exertion of a large force. Therefore, a need exists for handles equipped for use with medical device handles that include a control assembly for providing a mechanical advantage.

### SUMMARY

A medical device handle may comprise an actuator; a first pulley; and a second pulley. The second pulley may be movable relative to the first pulley along an axis extending between a first axle of the first pulley and a second axle of the second pulley. A pull member may be wrapped around the first pulley and the second pulley, and coupled to the actuator and a control member may be coupled to the second pulley. Movement of the second pulley along the axis may be configured to move the control member in a direction parallel to or coaxial with the axis.

Any of the devices or methods disclosed herein may include any of the following features in addition or in the alternative. The first pulley may be fixed relative to a body of the medical device handle. Each of the pull member and the control member may include at least one of a wire, a cable, or a thread. The axis extending between the first axle of the first pulley and the second axle of the second pulley may be approximately parallel to a longitudinal axis of the medical device handle. The control member may be configured to actuate an end effector at a distal end of a shaft extending from the medical device handle. The first pulley may be rotatable about the first axle, and the second pulley may be rotatable about the second axle. A resilient member may extend between the first pulley and the second pulley. The resilient member may include a spring. The medical device handle may be configured to transition between a first configuration, in which the first pulley and the second pulley are separated by a first distance, and a second configuration, in which the first pulley and the second pulley are separated by a second distance. The second distance may be smaller than the first distance. The resilient member may be configured to exert a restoring force on the second pulley when the medical device handle is in the second configuration. The first pulley and the second pulley may confer a mechanical advantage, such that a force exerted by the actuator on the pull member may be smaller than a force exerted by the second pulley on the control member. The actuator may include at least one finger loop. The second pulley may be coupled to the control member by a coupler. The coupler may be affixed to a face of the second pulley. Movement of the second pulley along the axis by a distance may be configured to move the control member by the distance.

In another example, a medical device handle may comprise an actuator; a first pulley; a second pulley; a pull member wrapped around the first pulley and the second pulley, and coupled to the actuator; and a control member coupled to the second pulley. The medical device handle may be configured to transition from a first configuration, in which the first pulley and the second pulley are separated by a first distance, to a second configuration, in which the first pulley and the second pulley are separated by a second distance. The second distance may be smaller than the first distance.

Any of the devices or methods disclosed herein may include any of the following features, in addition or in the alternative. Transitioning the medical device handle from the first configuration to the second configuration may cause the control member to move proximally. Movement of the control member may be configured to actuate an end effector at a distal end of a shaft extending from the medical device handle. A resilient member may extend between the first pulley and the second pulley.

In another example, a medical device handle may comprise an actuator; a first pulley; a second pulley; a pull member wrapped around the first pulley and the second pulley, and coupled to the actuator; and a control member coupled to the second pulley. The actuator may be configured to move the pull member proximally so as to move the second pulley proximally relative to the first pulley, thereby moving the control member proximally.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts an exemplary medical device.

FIG. 2 depicts a control assembly of the medical device of FIG. 1

FIG. 3A depicts the control assembly of FIG. 2 in a first configuration.

FIG. 3B depicts the control assembly of FIG. 2 in a second configuration.

### DETAILED DESCRIPTION

A medical device, such as an endoscopic medical device, may include a handle and a shaft, extending distally from the handle. The shaft of the medical device may be inserted into a working channel of another medical device (e.g., an endoscope or other type of scope). The handle may remain outside of a subject's body, so that it may be manipulated by an operator. The handle may include an actuator, such as one or more finger loops (e.g., at least one finger loop) and/or a slider that are slidable along a shaft of the handle. The actuator may be coupled to a control member (e.g., a control wire). Thus, moving the actuator proximally or distally may move the control member proximally or distally to actuate the medical device. For example, the actuator may be used to open/close jaws or blades, extend/retract a snare, a needle, a basket, or other device, deploy a medical device, staple tissue, cut tissue, or perform any other relevant action. The shaft of the medical device may pass through one or more tortuous body lumens when used alone or along with an endoscope or other type of scope. The tortuosity and/or a length of the shaft may require exertion of a large force by an operator on the actuator in order to move the actuator and actuate the medical device.

In order to decrease a force required by an operator to actuate the medical device, the handle may include one or more pulleys. For example, the handle may include a fixed pulley and a movable pulley. The movable pulley may be coupled to the control member. The actuator may be coupled to a pull member (e.g., a wire, a string, etc.) that is wrapped around the pulleys. As the actuator is moved proximally, the movable pulley may move proximally, thereby moving the control member proximally and actuating the medical device. A spring between the fixed pulley and the movable pulley may restore the movable pulley to an unactuated position (i.e., move the movable pulley distally) when the actuator is moved distally and/or released. The pulleys may decrease a force that is required to be exerted on the actuator in order to move the control member proximally.

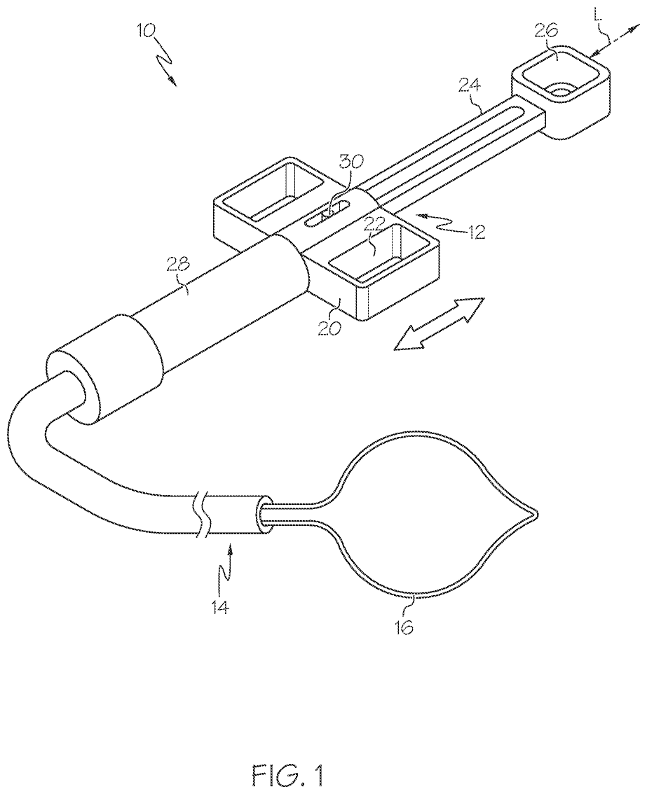

FIG. 1 depicts an exemplary medical device 10, which may include a handle 12 and an insertion portion/shaft 14. Handle 12 may be coupled to a shaft (not depicted), which may terminate in a distal tip. Handle 12, as discussed below, may be used to actuate an end effector 16. End effector 16 may include any type of assembly at a distal tip of a medical device, including, for example, a snare (shown in FIG. 1), a basket, a balloon, a stent delivery system, forceps, a stapler, a needle, a cautery device, a suturing device, an agent delivery system, a patch delivery system, or any other example. A longitudinal axis L of handle 12 may extend along a proximal/distal direction.

Handle 12 may include an actuator 20. As shown in FIG. 1, actuator 20 may include a slider having finger loops 22. Such a configuration is merely exemplary, and other configurations of actuator 20 may be utilized (e.g., a plunger, a slider without finger loops, or a lever). Actuator 20 may be movable proximally and distally (in the direction shown by the arrows of FIG. 1) along a shaft 24 of handle 12. Shaft 24 may include a thumb loop 26 at a proximal end thereof. Actuator 20 may be movable proximally until it reaches thumb loop 26 or until it reaches an alternative stop. In some examples, a configuration of actuator 20 in FIG. 1 may be a configuration in which actuator 20 is in a distalmost possible position. For example, a proximal portion of a body 28 of handle 12 may serve as a distal stop for actuator 20, preventing further distal movement of actuator 20. Shapes and relative sizes of actuator 20, shaft 24, and body 28 are merely exemplary, and other arrangements are contemplated within the scope of this disclosure. Although actuator 20 is depicted as an actuator for manual actuation by an operator, it will be appreciated that actuator 20 additionally or alternatively may be configured to be actuated robotically.

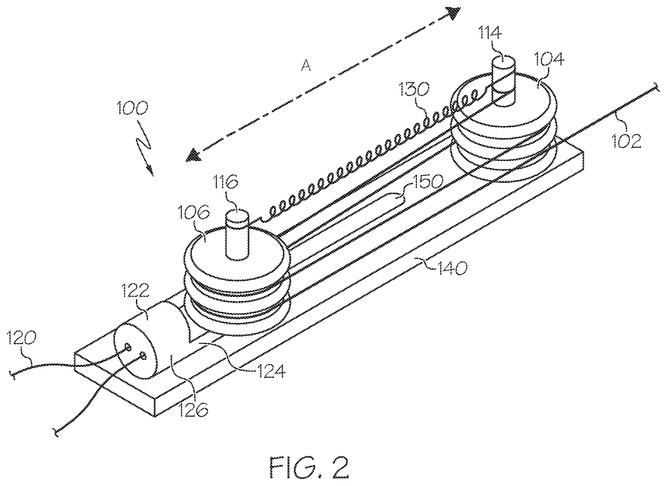

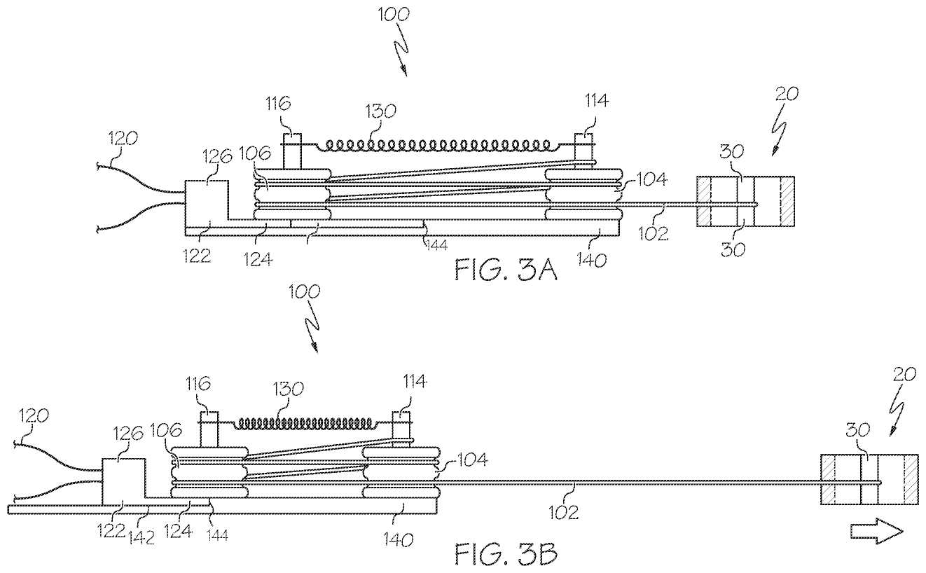

Actuator 20 may include an attachment 30 for coupling actuator 20 to a control assembly 100 (discussed in detail below with respect to FIGS. 2-3B). For example, as discussed below, attachment 30 may couple actuator 20 to a wire, a string, a thread, a cable, or other structure, either directly or indirectly. Attachment 30 may include a pin, a post, a rivet, a crimp, a clamp, an adhesive, a tie, a snap, or other structure. As actuator 20 moves proximally or distally, a portion of the wire, the string, or other structure coupled to actuator via attachment 30 may also move proximally or distally, respectively. As discussed below, movement of the wire, the string, or other structure may cause actuation of end effector 16 (e.g., in the case of the snare shown, opening/closing of the snare) via other elements of handle 12.

FIG. 2 depicts control assembly 100. FIG. 3A depicts control assembly 100 in a first configuration, and FIG. 3B depicts control assembly 100 in a second configuration. Elements of control assembly 100 may be disposed within body 28 of handle 12 or within other portions of handle 12 and/or shaft 14. Control assembly 100 may include a pull wire 102 or other type of pull member. Although the term “pull wire” is used herein, it will be appreciated that pull wire 102 may include at a thread, a string, a cable, and/or other structure (e.g., at least one of a wire, a thread, a cable, or a string). As discussed above and in further detail below, pull wire 102 may be coupled to actuator 20 (e.g., via attachment 30, as shown in FIGS. 3A and 3B). In FIGS. 3A and 3B, actuator 20 is shown in a cross-section taken along longitudinal axis L of handle 12 (along a proximal/distal direction).

Control assembly 100 also may include a first pulley 104 and a second pulley 106. In some examples, first pulley 104 may be proximal of second pulley 106 (i.e., second pulley 106 may be distal of first pulley 104). First pulley 104 may be axially (i.e., along longitudinal axis L of device 10/handle 12) and laterally (i.e., transverse to longitudinal axis L of device 10/handle 12) fixed relative to body 28 of handle 12 (and/or, for example, a housing of handle 12). First pulley 104 may be rotatable about a first axle 114, which may extend approximately perpendicularly to longitudinal axis L. Alternatively, first pulley 104 may be unable to rotate about first axle 114. In some examples, first axle 114 or another element of first pulley 104 may be fixedly coupled to a base 140, allowing rotation of first pulley 104 about first axle 114. Base 140 may include a portion of a housing of handle 12 (e.g., a housing of body 28) or may be a separate element within the housing of handle 12. Fixing first axle 114 or another element of first pulley 104 to base 140 may inhibit/prevent first pulley 104 from moving axially and laterally, as discussed above.

Second pulley 106 may be rotatable about a second axle 116, which may extend approximately parallel to first axle 114, and approximately perpendicularly to longitudinal axis L. Alternatively, second pulley 106 may be unable to rotate about second axle 116. Second pulley 106 may be movable along an axis A that extends between first axle 114 and second axle 116 (while first pulley 104 may be fixed relative to body 28 or another portion of handle 12 along the axis A extending between first axle 114 and second axle 116, as discussed above). For example, axis A may be coaxial with or parallel to longitudinal axis L, and second pulley 106 may be axially movable relative to first pulley 104, along longitudinal axis L or parallel to longitudinal axis L. Alternatively, axis A may be transverse to longitudinal axis L.

Second pulley 106 may be constrained from moving laterally, in a direction transverse to (a) axis A and/or (b) longitudinal axis L. For example, as shown in FIG. 2, base 140 may include a slot 150. Slot 150 may extend approximately parallel to or coaxial with longitudinal axis L and/or approximately parallel to or coaxial with axis A. Second axle 116, or another element of second pulley 106, may be movable within slot 150. Slot 150 may constrain second pulley 106 from moving laterally. Slot 150 is merely exemplary, and additional or alternative structures (e.g., sidewalls, guiderails, etc.) may be utilized to constrain second pulley 106 so that it moves only in a desired direction (e.g., axially and not laterally).

One or more control wires 120 or other types of control members may be coupled to second pulley 106 via a coupler 122. Although the term “control wire” is used herein, it will be appreciated that control wire(s) 120 may additionally or alternatively include cables, threads, strings, or other structures along an entirety or a portion thereof. FIGS. 2-3B depict two control wires 120. Each of control wires 120 may be, for example, coupled to a leg of a snare forming end effector 16. Proximal movement of control wires 120 may cause the legs of the snare to collapse and be retracted within a sheath of shaft 14. Distal movement of control wires 120 may cause the legs of the snare to expand and to extend distally of shaft 14. However, a single control wire 120 may be used to actuate an end effector 16, or more than two control wires 120 may be used to actuate an end effector 16. A number and arrangement of control wire(s) 120 may depend upon the type of end effector 16. Control wire(s) 120 may be separate elements from pull wire 102 (i.e., control wire(s) 120 may not be integrally formed with pull wire 102).

Coupler 122 may be coupled to second pulley 106 (e.g., fixedly coupled to second pulley 106). Control wire(s) 120 also may be coupled to coupler 122 (e.g., via crimping, adhesive, screws, rivets, clamps, interference fit, knots or other ties, or any other suitable mechanism). As shown in the Figures, coupler 122 may include an arm 124 and a body 126. Arm 124 may be coupled/affixed to a face/side of second pulley 106 (a portion of second pulley 106 on which pull wire 102 does not wrap around). Body 126 may be distal to arm 124 and distal to second pulley 106. Arm 124 may be thinner than body 126 because it may be desirable to have a thinner profile of coupler 122 along a portion of coupler 122 (i.e., arm 124) that extends along second pulley 106. A portion of base 140 may include a recess 142 for receiving arm 124 as it moves proximally along base 140 (see FIG. 3A). A proximal edge 144 of recess 142 may serve as a stop for arm 124, such that when a proximal end of arm 124 contacts proximal edge 144, arm 124, and second pulley 106, may be prevented from moving further proximally. A configuration of coupler 122 is merely exemplary, and control wire 120 may be coupled to second pulley 106 in any desired fashion. For example, one or more arms may extend from second axle 116 and may couple to control wire(s) 120.

Pull wire 102 may be wrapped around first pulley 104 and second pulley 106 in any suitable fashion. First pulley 104 and second pulley 106 may form a compound pulley system. Pull wire 102, first pulley 104, and second pulley 106 may be arranged in any compound pulley arrangement that is known or becomes known. Although the Figures depict first pulley 104 and second pulley 106 as being arranged linearly (longitudinal axis L extends through or approximately parallel with first axle 114 and second axle 116), such an arrangement is merely exemplary. First pulley 104 and second pulley 106 may be offset from one another in a lateral (or other) direction. Furthermore, although two pulleys 104, 106 are depicted, alternative numbers of pulleys may be utilized (e.g., three, four, or five pulleys).

Pull wire 102 may be wrapped around pulleys 104, 106 in any suitable fashion, as discussed below. As pull wire 102 is moved proximally, to transition control assembly 100 to the second configuration of FIG. 3B (e.g., via actuator 20 being moved proximally by an operator), pull wire 102 may interact with first pulley 104 and second pulley 106 (e.g., by causing first pulley 104 and/or second pulley 106 to rotate about axles 114, 116, respectively, or by moving with respect to first pulley 104 and/or second pulley 106) to cause second pulley 106 to move proximally. Proximal movement of second pulley 106 may, in turn, exert a force on control wire(s) 120 (e.g., via coupler 122), moving control wire(s) 120 proximally. Such proximal movement of control wire(s) 120 may actuate end effector 16 (e.g., close the snare depicted in FIG. 1 or perform any other desired action).

Control assembly 100 may be configured such that movement of second pulley 106 by an amount may be configured to move control wire(s) 120 (e.g., a proximal end of control wire(s) 120) by an equal amount. In other words, a change in a distance between first pulley 104 and second pulley 106 corresponds to moving control wire(s) 120 by the same amount (equal to the change in distance). For example, in the first configuration of FIG. 3A, first pulley 104 and second pulley 106 may be separated by a first distance. In FIG. 3B, as shown by the arrow, actuator 20 may be moved proximally, which may, in turn, move a proximal end of pull wire 102 proximally. This may move second pulley 106 along axis A (e.g., proximally), such that first pulley 104 and second pulley 106 are separated by a second distance that is smaller than the first distance of the first configuration. A difference between the first distance and the second distance may correspond to an amount that control wire(s) 120 is moved proximally (e.g., that a proximal end of control wire(s) 120 is moved proximally).

Pulleys 104, 106 may confer a mechanical advantage. In other words, a force applied to actuator 20 may be smaller than a force transmitted to control wire(s) 120. Pulleys 104, 106 may amplify the force applied to actuator 20. In other words, for a given force that is required to move control wire(s) 120, a smaller force may be required on actuator 20. During a medical procedure, shaft 14 of device 10 may be inserted in a tortuous body lumen or otherwise subjected to forces that require a larger force to be exerted on control wire(s) 120 than when shaft 14 is straight. In the absence of pulleys 104, 106, the larger force that would be required to be applied to actuator 20 in such a configuration may pose difficulties for operators. The mechanical advantage provided by pulleys 104, 106 may decrease the amount of force that is required to be applied to actuator 20. Such mechanical advantage exists when shaft 14 is in both straight and bent configurations. The mechanical advantage may be particularly advantageous when shaft 14 is bent (e.g., within a tortuous body lumen) or when a force required to move control wire(s) 120 otherwise increases.

A resilient member 130 may extend between first pulley 104 and second pulley 106. For example, resilient member 130 may be fixed at a first end to first axle 114 of first pulley 104 and at a second end to second axle 116 of second pulley 106. Resilient member 130 may be biased into a configuration that corresponds to an unactuated configuration of end effector 16 (e.g., expanded, retracted, open, closed, or non-delivery, etc. configuration). Thus, when actuator 20 is released, resilient member 130 may exert a restoring force to return second pulley 106 (and control wire 120) to a configuration to which they are biased (e.g., the first configuration of FIG. 3A). For example, when actuator 20 is moved proximally in order to actuate device 10, as shown in FIG. 3B, thereby moving second pulley 106 proximally, and actuator 20 is subsequently released or moved distally, resilient member 130 may exert a distal force on second pulley 106 and move it distally. Although resilient member 130 is depicted as a coil spring, it will be appreciated that alternative structures may be used (e.g., a leaf spring, an air compression piston, or structures with shape memory). Alternatively, resilient member 130 may be omitted or may be positioned between alternative structures (e.g., between second pulley 106 and a housing of handle 12). Alternatives to resilient member 130 may include further actuators (e.g., an actuator attached to second axle 116 and extending through a housing of handle 12), or other structures for moving second pulley 106. Additionally or alternatively, actuator 20 may include a lock for retaining second pulley 106/control wire(s) 120/end effector 16 in a desired configuration (e.g., a configuration in which second pulley 106/control wire(s) 120 has been moved proximally).

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device handle comprising:
an actuator;
a first pulley;
a second pulley, wherein the second pulley is movable relative to the first pulley along an axis extending between a first axle of the first pulley and a second axle of the second pulley;
a pull member wrapped around the first pulley and the second pulley, and coupled to the actuator; and
a control member coupled to the second pulley, wherein movement of the second pulley along the axis is configured to move the control member in a direction parallel to or coaxial with the axis.

2. The medical device handle of claim 1, wherein the first pulley is fixed relative to a body of the medical device handle.

3. The medical device handle of claim 1, wherein each of the pull member and the control member includes at least one of a wire, a cable, or a thread.

4. The medical device handle of claim 1, wherein the axis extending between the first axle of the first pulley and the second axle of the second pulley is approximately parallel to a longitudinal axis of the medical device handle.

5. The medical device handle of claim 1, wherein the control member is configured to actuate an end effector at a distal end of a shaft extending from the medical device handle.

6. The medical device handle of claim 1, wherein the first pulley is rotatable about the first axle, and wherein the second pulley is rotatable about the second axle.

7. The medical device handle of claim 1, further comprising a resilient member extending between the first pulley and the second pulley.

8. The medical device handle of claim 7, wherein the resilient member includes a spring.

9. The medical device handle of claim 7, wherein the medical device handle is configured to transition between a first configuration, in which the first pulley and the second pulley are separated by a first distance, and a second configuration, in which the first pulley and the second pulley are separated by a second distance, wherein the second distance is smaller than the first distance.

10. The medical device handle of claim 9, wherein the resilient member is configured to exert a restoring force on the second pulley when the medical device handle is in the second configuration.

11. The medical device handle of claim 1, wherein the first pulley and the second pulley confer a mechanical advantage, such that a force exerted by the actuator on the pull member is smaller than a force exerted by the second pulley on the control member.

12. The medical device handle of claim 1, wherein the actuator includes at least one finger loop.

13. The medical device handle of claim 1, wherein the second pulley is coupled to the control member by a coupler.

14. The medical device handle of claim 13, wherein the coupler is affixed to a face of the second pulley.

15. The medical device handle of claim 1, wherein movement of the second pulley along the axis by a distance is configured to move the control member by the distance.

16. A medical device handle comprising:
an actuator;
a first pulley;
a second pulley;
a pull member wrapped around the first pulley and the second pulley, and coupled to the actuator; and
a control member coupled to the second pulley;
wherein the medical device handle is configured to transition from a first configuration, in which the first pulley and the second pulley are separated by a first distance, to a second configuration, in which the first pulley and the second pulley are separated by a second distance, wherein the second distance is smaller than the first distance.

17. The medical device handle of claim 16, wherein transitioning the medical device handle from the first configuration to the second configuration causes the control member to move proximally.

18. The medical device handle of claim 17, wherein movement of the control member is configured to actuate an end effector at a distal end of a shaft extending from the medical device handle.

19. The medical device handle of claim 16, further comprising a resilient member extending between the first pulley and the second pulley.

20. A medical device handle comprising:
an actuator;
a first pulley;
a second pulley;
a pull member wrapped around the first pulley and the second pulley, and coupled to the actuator; and
a control member coupled to the second pulley;
wherein the actuator is configured to move the pull member proximally so as to move the second pulley proximally relative to the first pulley, thereby moving the control member proximally.
